(19) United States
(12) Patent Application Publication
(10) Pub. No.: US 2018/0015042 A1
(43) Pub. Date: Jan. 18, 2018

(54) PHARMACEUTICAL COMPOSITION FOR THE TREATMENT OF OBESITY

(71) Applicant: Intas Pharmaceuticals Ltd., Ahmedabad, Gujarat (IN)

(72) Inventors: Ashish SEHGAL, Ahmedabad (IN); Abhishek JAIN, Ahmedabad (IN); Malay PATEL, Ahmedabad (IN); Sunil BORUDE, Ahmedabad (IN); Himanshukumar PATEL, Ahmedabad (IN); Vinod DUBE, Ahmedabad (IN)

(21) Appl. No.: 15/548,985

(22) PCT Filed: Feb. 5, 2016
(86) PCT No.: PCT/IB2016/050595

§ 371 Date: Aug. 4, 2017

(30) Foreign Application Priority Data
Feb. 7, 2015 (IN) 406/MUM/2015

(51) Int. Cl. A61K 9/24; A61K 47/14; A61K 31/485; A61K 47/26; A61K 31/137

(52) U.S. Cl. CPC A61K 9/209; A61K 47/26; A61K 31/137; A61K 31/485; A61K 47/14

(57) ABSTRACT

The present invention relates to a layered pharmaceutical composition comprising: a first layer of sustained-release bupropion or its pharmaceutically acceptable salts, a second layer of sustained-release naltrexone or its pharmaceutically acceptable salts, with pharmaceutically acceptable excipients, and an intermediate inert layer that provides a time-controlled disintegration to allow separation of two drug layers and maintain their physical integrity as a single tablet. It further provides the methods for preparing the said pharmaceutical compositions for the treatment of obesity-related conditions.

### RELATED APPLICATIONS

This application is related to Indian Provisional Application 406/MUM/2015 filed 7 Feb. 2015 and is incorporated herein in its entirety.

### FIELD OF THE INVENTION

The present invention relates to a layered pharmaceutical composition comprising a first layer of sustained-release bupropion or its pharmaceutically acceptable salts and a second layer of sustained-release naltrexone or its pharmaceutically acceptable salts with suitable pharmaceutically acceptable excipients for the treatment of obesity-related conditions. It further provides the methods for preparing the said pharmaceutical compositions.

### BACKGROUND OF THE INVENTION

Obesity is generally referred as a disorder characterized by the excess accumulation of fat in the body resulting in an increased body weight and body fat percentage. Obesity has been recognized as one of the leading causes of disease and is emerging as a global problem. Obesity is a chronic disease associated with high morbidity and mortality, caused by adipose tissue accumulation due to disrupted regulation of energy balance or hyper nutrition. There are currently 250 million obese people in the world, and it is estimated that about 300 million people worldwide will be obese by the year 2025.

The BMI (Body Mass Index) value has been used as a standard measurement of obesity and over-weight. There has been reported that the BMI values over 25 and 30 indicate over-weight and obesity respectively in case of western people and the BMI value above 23 indicates over-weight and the precaution of adult disease.

There have been several methods to treat obesity, for example, diet therapy or exercise therapy, however, those methods often result in failure because of genetic factor such as personal differences in respect to appetite, favor to high-fat food and metabolism of fat formation. Therefore, there exists a need for therapy to promote reducing body weight other than classical approach methods.

The drugs for the treatment of obesity include, not limiting examples, such as Orlistat, Lorcaserin, Sibutramine, Rimonabant, Metformin, Exenatide, Pramlintide, combination of Phentermine/Topiramate, combination of Naltrexone/Bupropion, combination of Bupropion/Zonisamide, GT389-255 (Investigational drug), Diethylpropion, Liraglutide, Methamphetamine, Phendimetrazine, Benzphetamine. Such drugs can be administered by oral or parenteral route of administration by a person with ordinary skill in the art.

Bupropion is a weak norepinephrine-dopamine reuptake inhibitor (NDRI) and may act as a releasing agent of dopamine and norepinephrine. The IUPAC name of Bupropion is (±)-2-(tert-Butylamino)-1-(3-chlorophenyl) propan-1-one. The chemical structure of Bupropion is shown in formula below:

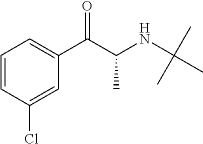 [SMILES: C[C@@H](NC(C)(C)C)C(=O)c1cccc(Cl)c1]

Naltrexone is an opioid receptor antagonist used primarily in the management of alcohol dependence and opioid dependence. The IUPAC name of Naltrexone is 17-(cyclopropylmethyl)-4,5α-epoxy-3,14-dihydroxymorphinan-6-one. The chemical name of Naltrexone is shown in formula below:

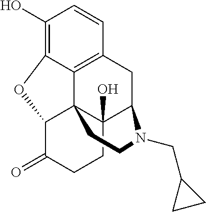

Currently, the combination of Bupropion/Naltrexone is available as an extended-release tablet)(CONTRAVE®) marketed by Orexigen Therapeutics. CONTRAVE® is a round, bi-convex, film coated, extended release trilayer tablet. Each trilayer tablet comprises two drug layers, containing the drug and excipients, separated by rapidly dissolving inert layer. Each trilayer tablet contains 8 mg of naltrexone hydrochloride and 90 mg of bupropion hydrochloride.

The composition and the use of CONTRAVE® tablet is disclosed in U.S. Pat. Nos. 7,375,111, 7,462,626, 8,088,786, 8,318,788, 8,722,085, 8,815,889, and 8,916,195. However, the CONTRAVE® is a complex trilayer tablet wherein the middle layer essentially needs to dissolve rapidly within a time period of 30 minutes to separate the upper and lower drug layers. The said compositions may cause problems of reproducibility and uncertainty regarding predictability of disintegration and dissolution. Further, there exists a need to develop a simple composition from economical point of view with a lesser number of unit operations for the bulk production. Hence, there exists a strong need for the development of a simple and stable pharmaceutical composition to overcome the problems of the prior art.

### OBJECTS OF THE INVENTION

The primary object of the invention is to provide a layered pharmaceutical composition comprising: a first layer of sustained-release bupropion or its pharmaceutically acceptable salts, a second layer of sustained-release naltrexone or its pharmaceutically acceptable salts, with suitable pharmaceutically acceptable excipients, and an intermediate inert layer that provides a time-controlled disintegration to allow separation of two drug layers and maintain their physical integrity as a single tablet.

Another object of the invention is to provide a process for the preparation of a layered pharmaceutical composition comprising a first layer of sustained-release bupropion or its pharmaceutically acceptable salts and a second layer of sustained-release naltrexone or its pharmaceutically acceptable salts, with suitable pharmaceutically acceptable excipients, and an intermediate inert layer that provides a time-controlled disintegration to allow separation of two drug layers and maintain their physical integrity as a single tablet.

### SUMMARY OF THE INVENTION

In a first embodiment, the invention relates to a layered pharmaceutical composition comprising: a first layer of sustained-release bupropion or its pharmaceutically acceptable salts, a second layer of sustained-release naltrexone or its pharmaceutically acceptable salts, with suitable pharmaceutically acceptable excipients, and an intermediate inert layer that provides a time-controlled disintegration to allow separation of two drug layers and maintain their physical integrity as a single tablet.

In a preferred embodiment, the invention relates to a trilayer tablet comprising a first layer of sustained-release bupropion or its pharmaceutically acceptable salts and a second layer of sustained-release naltrexone or its pharmaceutically acceptable salts, wherein the intermediate layer comprises mannitol with suitable pharmaceutically acceptable excipients.

In a preferred embodiment, the invention relates to a trilayer tablet comprising a first matrix layer of sustained-release bupropion or its pharmaceutically acceptable salts and a second matrix layer of sustained-release naltrexone or its pharmaceutically acceptable salts, wherein the intermediate layer comprises glyceryl behenate with suitable pharmaceutically acceptable excipients.

In another embodiment, the invention relates to a process for the preparation of a layered pharmaceutical composition comprising bupropion or its pharmaceutically acceptable salts and naltrexone or its pharmaceutically acceptable salts, and an intermediate inert layer that provides a time-controlled disintegration to allow separation of two drug layers and maintain their physical integrity as a single tablet. The said layered pharmaceutical composition can be prepared by direct compression, dry granulation, wet granulation or pelletization method.

### DETAILED DESCRIPTION

The present invention relates to a layered pharmaceutical composition comprising: a first layer of sustained-release bupropion or its pharmaceutically acceptable salts, a second layer of sustained-release naltrexone or its pharmaceutically acceptable salts, and an intermediate inert layer that provides a time-controlled disintegration to allow separation of two drug layers and maintain their physical integrity as a single tablet.

The term “sustained-release” refers to a pharmaceutical composition such that its dissolution profile is extended over a longer period of time than that of an immediate release composition.

In a preferred embodiment, the sustained-release composition can be obtained with suitable pharmaceutically acceptable excipients, by either matrix layer or multiple-unit pellet system (MUPS).

The term “matrix layer” refers to an active drug layer comprising at least one drug with at least one rate-controlling agent such that it produces sustained-release drug matrix layer, which can be incorporated into a layered pharmaceutical composition of the present invention.

The term “multiple-unit pellet system (MUPS)” refers to an agglomerate of pellets comprising at least one drug with suitable pharmaceutically acceptable excipients such that it produces sustained-release drug pellets, which can be incorporated into a layered pharmaceutical composition of the present invention.

The “layered pharmaceutical composition” includes at least two or more active drug layers with an intermediate inert layer. In a preferred embodiment, it comprises a trilayer tablet.

In a preferred embodiment, the layered pharmaceutical composition comprises a first layer comprising between about 50 mg and about 200 mg of sustained-release bupropion or its pharmaceutically acceptable salts and a second layer comprising between about 2 mg and about 35 mg of sustained-release naltrexone or its pharmaceutically acceptable salts, and an intermediate inert layer comprising mannitol or glyceryl behenate with suitable pharmaceutically acceptable excipients.

The intermediate inert layer comprises mannitol or glyceryl behenate with suitable pharmaceutically acceptable excipients to provide a time-controlled disintegration in less than 5 minutes. Preferably the time-controlled disintegration occurs in less than 1 minute.

Not bound to any theory, the term “time-controlled disintegration” for the purpose of the invention refers to the physical breakdown of layered pharmaceutical composition to allow separation of two drug layers and maintain their physical integrity as a single tablet.

Not bound to any theory, the term “physical integrity” for the purpose of the invention refers to the tablet structure in a substantially intact form as a single tablet for the time period during which the drug is released.

The suitable pharmaceutically acceptable excipients that can be incorporated into sustained-release pharmaceutical compositions include diluents, binders, rate-controlling agents, stabilizers, wetting agents, lubricants, glidants and coating excipients. Preferably the non-limiting examples, includes L-Cysteine HCl, Microcrystalline Cellulose (MCC), silicified microcrystalline cellulose, PROSOLVE SMCC 90 (i.e. Silicified microcrystalline cellulose composed of 98% microcrystalline cellulose and 2% colloidal silicon dioxide), Lactose Monohydrate, Hypromellose (HPMC), Hydroxypropyl Cellulose (HPC), Ethyl cellulose, Crospovidone, croscarmellose sodium, sodium starch glycolate (SSG), mannitol, glyceryl behenate, Dibasic calcium phosphate dihydrate, Magnesium stearate, Colloidal Silicon Dioxide, Edetate Disodium, Polyethylene glycol (PEG), hydrogenated vegetable oil, or other conventional tablet excipients thereof.

The rate-controlling agents incorporated into any of the drug layers of the sustained-release pharmaceutical compositions, are within the concentration of 1-50% W/W. Preferably, the “rate-controlling agents” of the present invention includes, non-limiting examples, such as Hypromellose (HPMC), Hydroxypropyl Cellulose (HPC), Ethyl cellulose or mixtures thereof.

In another embodiment, the invention relates to a layered pharmaceutical composition, wherein the first layer comprises between about 50 mg and about 200 mg of sustained-release bupropion or its pharmaceutically acceptable salts, a second layer comprises between about 2 mg and about 35 mg of sustained-release naltrexone or its pharmaceutically acceptable salts, and an intermediate inert layer, wherein the naltrexone dissolution profile in a dissolution test of USP Apparatus 2 Paddle Method at 50 rpm in a dissolution medium of water is:

a) between 40 to 80% of naltrexone released in one hour;

b) between 60 to 90% of naltrexone released in two hours.

c) at least 99% % of naltrexone released in 8 hours.

In another embodiment, the invention relates to the stability of the layered pharmaceutical compositions, wherein the impurity profile is within the prescribed limits. The Examples 5 and 6 describes the impurities of Bupropion Layer and Naltrexone Layer with their measured limit.

In another embodiment, the invention relates to a method of treating overweight or obesity, comprising a layered pharmaceutical composition comprises, wherein about 180 mg of said sustained-release formulation of bupropion or a pharmaceutically acceptable salt thereof is administered twice daily, and about 16 mg of said sustained-release formulation of naltrexone or a pharmaceutically acceptable salt thereof is administered twice daily.

In another embodiment, the invention relates to a process for the preparation of a layered pharmaceutical composition comprising bupropion or its pharmaceutically acceptable salts and naltrexone or its pharmaceutically acceptable salts with suitable pharmaceutically acceptable excipients. The said layered pharmaceutical composition can be prepared by direct compression, dry granulation, wet granulation or pelletization method.

In order to further illustrate the present invention, the following examples are provided for the purpose of clarity of understanding. However, it is not intended in any way to limit the scope of present invention and it is readily apparent to those of ordinary skill in the art in light of the teachings of this invention that certain changes and modifications may be made thereto without departing from the scope of the invention.

### EXAMPLE 1

### Trilayer Tablet (Bupropion HCl Sustained-Release Matrix Layer; Naltrexone HCl Sustained-Release Matrix Layer and an Intermediate Inert Layer

| | | |
|---|---|---|
| Ingredient | Qty (mg/tablet) | % W/W |

Drug Layer - I

| | | |
|---|---|---|
| Bupropion hydrochloride | 90.0 | 5-40 |
| L-Cysteine HCl | 4.0 | 0-20 |
| Microcrystalline Cellulose (MCC) | 70.0 | 5-50 |
| Hydroxypropyl Cellulose (HPC)/ | 77.0 | 1-50 |
| Hypromellose | | |
| Magnesium stearate | 6.0 | 0.5-10 |
| Total wt. (1^{st} layer) in mg | 247.0 | |

Intermediate Inert Layer

| | | |
|---|---|---|
| Microcrystalline Cellulose (MCC) | 84.0 | 1-50 |
| Mannitol | 80.0 | 1-50 |
| Crospovidone | 10.0 | 1-10 |
| Magnesium stearate | 5.0 | 0.5-10 |
| FD & C Blue # 2 Aluminium lake | 1.0 | 0.5-2 |
| Total wt. (2^{nd} layer) in mg | 180.0 | |

Drug Layer - II

| | | |
|---|---|---|
| Naltrexone hydrochloride | 8.0 | 1-25 |
| Microcrystalline Cellulose (MCC) | 62.0 | 3-30 |
| Hypromellose K4M (HPMC K4M) | 60.0 | 3-30 |
| Hydroxypropyl Cellulose (HPC) | 17.0 | 1-30 |
| Edetate Disodium | 4.0 | 0.5-10 |
| Colloidal Silicon Dioxide | 14.0 | 0.5-10 |
| Lactose Monohydrate | 62.0 | 3-30 |
| Magnesium stearate | 6.0 | 0.5-10 |
| Total wt. (3^{rd} layer) in mg | 233.0 | |
| Total wt. (1^{st} + 2^{nd} + 3^{rd} layer) in mg | 660.0 | |

Film Coating

| | |
|---|---|
| Opadry-II Blue in mg | 20.0 |
| Total weight in mg | 680.0 |

Manufacturing Process:
(i) Weigh all the ingredients required for the preparation of drug layer-I,
(ii) Sift all ingredients of drug layer -I through 30 # sieve and load in granulator,
(iii) Granulate the blend of the sifted ingredients for an appropriate time,
(iv) Dry the granules appropriately till LOD less than 1.5%.
(v) Sift the dried granules from 20# sieve and add lubricant to the dry granules.
(vi) Weigh all the ingredients required for the preparation of inert layer-II,
(vii) Sift all ingredients of drug layer -II through 40 # sieve and blend for 5 minutes in a blender.
(viii) Weigh all the ingredients required for the preparation of drug layer-III,
(ix) Sift all ingredients of drug layer -III through 30 # sieve and load in granulator,
(x) Granulate the blend of the sifted ingredients for an appropriate time,
(xi) Dry the granules appropriately till LOD less than 1.5%.
(xii) Sift the dried granules from 20# sieve and add lubricant to the dry granules.
(xiii) Blend of all the three layers is compressed to prepare a trilayer tablet.
(xiv) Optionally film coat the compressed tablets.

### EXAMPLE 2

### Trilayer Tablet (Bupropion HCl Sustained-Release Matrix Layer; Naltrexone HCl Sustained-Release Matrix Layer and an Intermediate Inert Layer

| | | |
|---|---|---|
| Ingredient | Qty (mg/tablet) | % W/W |

Drug Layer - I

| | | |
|---|---|---|
| Bupropion hydrochloride | 90.0 | 5-40 |
| L-Cysteine HCl | 4.0 | 1-20 |
| Microcrystalline Cellulose (MCC) | 70.0 | 5-50 |
| Hydroxypropyl Cellulose (HPC) | 77.0 | 1-50 |
| Magnesium stearate | 6.0 | 0.5-10 |
| Total wt. (1^{st} layer) in mg | 247.0 | |

Intermediate Inert Layer

| | | |
|---|---|---|
| Glyceryl dibehenate | 50.0 | 1-50 |
| Dibasic calcium phosphate dihydrate | 110.0 | 5-50 |
| Colloidal Silicon Dioxide | 14.0 | 0.5-10 |
| Magnesium stearate | 5.0 | 0.5-5 |
| FD & C Blue # 2 Aluminium lake | 1.0 | 0.5-10 |
| Total wt. (2^{nd} layer) in mg | 180.0 | |

Drug Layer - II

| | | |
|---|---|---|
| Naltrexone hydrochloride | 8.0 | 1-25 |
| Microcrystalline Cellulose (MCC) | 82.0 | 10-50 |
| Hypromellose K4M (HPMC K4M) | 40.0 | 1-50 |
| Hydroxypropyl Cellulose (HPC) | 17.0 | 1-20 |
| Edetate Disodium | 4.0 | 0.5-10 |
| Colloidal Silicon Dioxide | 14.0 | 0.5-10 |
| Lactose Monohydrate | 62.0 | 1-25 |
| Magnesium stearate | 6.0 | 0.5-10 |
| Total wt. (3^{rd} layer) in mg | 233.0 | |
| Total wt. (1^{st} + 2^{nd} + 3^{rd} layer) in mg | 660.0 | |

Film Coating

| | |
|---|---|
| Opadry-II Blue in mg | 20.0 |
| Total weight in mg | 680.0 |

The pharmaceutical composition of Example 2 is prepared by the same manufacturing procedure as described for the Example 1.

### EXAMPLE 3

### Trilayer Tablet (Bupropion HCl Sustained-Release Matrix Layer; Naltrexone HCl Sustained-Release Matrix Layer and an Intermediate Inert Layer

Batch ASBNET1017 (Coated Tablets)

| | | |
|---|---|---|
| Sr. No | Ingredients | mg/tab |

Drug Layer - I

| | | |
|---|---|---|
| 1 | Bupropion HCl | 90 |
| 2 | MCC-112 | 156 |
| 3 | HPC | 20 |
| 4 | Cysteine HCl monohydrate | 8 |
| 5 | HPC | 15 |
| 6 | Purified Water | q.s. |
| 7 | Magnesium stearate | 6 |
| 8 | HPC | 25 |
| | Part 1 Avg. wt | 320 |

Intermediate Inert Layer

| | | |
|---|---|---|
| 1 | Microcrystalline Cellulose (MCC-112) | 56.5 |
| 2 | Mannitol | 58 |
| 3 | Crospovidone | 3 |
| 4 | Magnesium stearate | 2 |
| 5 | FD & C Blue # 2 Aluminium lake | 0.5 |
| | Part 2 Avg. wt | 120 |

Drug Layer - II

| | | |
|---|---|---|
| 1 | Naltrexone hydrochloride | 8 |
| 2 | Silicified MCC | 171 |
| 3 | Hypromellose K4M premier CR | 32 |
| 4 | Edetate Disodium | 5 |
| 5 | Magnesium stearate | 4 |
| | Part 3 Avg. Wt | 220 |
| | Total | 660 |

Film Coating

| | | |
|---|---|---|
| 1 | Opadry-II Blue in mg | 26 |
| 2 | Total weight in mg | 686 |

The manufacturing Process for Example 3 is same as that described for Example 1.

### EXAMPLE 4

### Dissolution Profile of Trilayer Tablet (as Described in Example 3)

| | Method | | | |
|---|---|---|---|---|
| | Paddle, 50 RPM, Sinker | | Paddle, 50 RPM, Sinker | |
| | Bupropion Part | | Naltrexone Part | |
| Time (Hr) | % Drug Release | RSD | % Drug Release | RSD |
| 0.5 | 30 | 6.94 | 45 | 24.87 |
| 1 | 44 | 10.45 | 64 | 17.75 |
| 2 | 63 | 12.73 | 82 | 10.37 |
| 3 | 77 | 13.01 | 91 | 6.28 |
| 4 | 86 | 11.63 | 96 | 4.62 |
| 6 | 96 | 7.82 | 100 | 4.58 |
| 8 | 100 | 4.37 | 102 | 3.98 |
| 12 | 102 | 1.56 | 102 | 4.13 |

The intermediate inert layer of the trilayer tablet provides a time-controlled disintegration (i.e. within 5 minutes) to allow separation of two drug layers and maintain their physical integrity as a single tablet.

The naltrexone dissolution profile in a dissolution test of USP Apparatus 2 Paddle Method at 50 rpm in a dissolution medium of water is:

a) between 40 to 80% of naltrexone released in one hour;

b) between 60 to 90% of naltrexone released in two hours; and

c) at least 99% % of naltrexone released in 8 hours.

The sustained-release formulation of naltrexone or a pharmaceutically acceptable salt thereof provides an in-vitro release rate of naltrexone in the dissolution test of at least 99% in 8 hours.

### EXAMPLE 5

### Stability Data of Trilayer Tablet

| | B. No ASBNET1017 - | | |
|---|---|---|---|
| | 1^{st} Sample | | |
| Related Substance | Initial | 1M ACC | 2M ACC |

Bupropion HCL Part

| | | | |
|---|---|---|---|
| Compound F (RRT 1.84) | 0.345 | 0.744 | 0.997 |
| Compound C (RRT 1.91) | ND | 0.021 | 0.028 |
| Single Maximum Impurity | 0.041 | 0.121 | 0.203 |
| Total Impurity (3.3%) | 0.544 | 1.341 | 2.618 |

Naltrexone HCL Part

| | | | |
|---|---|---|---|
| Impurity J (RRT 1.96) | ND | ND | ND |
| Impurity D | 0.093 | ND | 0.109 |
| Single Maximum Impurity | 0.383 | 0.359 | ND |
| Total Impurity | 0.732 | 0.684 | 0.109 |

Compound F: (1-(3-chlorophenyl)-1-hydroxy-2-propanone) - Limit is 2.5%.
Compound C: (1-(3-chlorophenyl)-2-hydroxy-1-propanone) - Limit is 0.5%.
Impurity J: 17-(Cyclopropylmethyl)-4,5-α-epoxy-14-hydroxy-3-methoxy morphinan-6-one.
Impurity D: 2,2′ Bisnaltrexone
RRT: Relative Retention Time
CRT: Controlled Room Temperature

The stability data indicated that the trilayer tablets remained stable since the impurity profile is within the prescribed limits.

### EXAMPLE 6

### Stability Data of Trilayer Tablet

| | B. No ASBNET1017 - | | |
|---|---|---|---|
| | 2^{nd} Sample | | |
| Related Substance | Initial | 1M ACC | 2M ACC |

Bupropion HCL Part

| | | | |
|---|---|---|---|
| Compound F (RRT 1.84) | 0.345 | 1.067 | 1.481 |
| Compound C (RRT 1.91) | ND | 0.022 | 0.037 |
| Single Maximum Impurity | 0.041 | 0.198 | 0.165 |
| Total Impurity (3.3%) | 0.544 | 2.191 | 3.884 |

Naltrexone HCL Part

| | | | |
|---|---|---|---|
| Impurity J (RRT 1.96) | ND | ND | ND |
| Impurity D | 0.093 | ND | 0.131 |
| Single Maximum Impurity | 0.383 | 0.511 | ND |
| Total Impurity | 0.732 | 0.912 | 0.131 |

Compound F: (1-(3-chlorophenyl)-1-hydroxy-2-propanone) - Limit is 2.5%.
Compound C: (1-(3-chlorophenyl)-2-hydroxy-1-propanone) - Limit is 0.5%.
Impurity J: 17-(Cyclopropylmethyl)-4,5-α-epoxy-14-hydroxy-3-methoxy morphinan-6-one.
Impurity D: 2,2′ Bisnaltrexone
RRT: Relative Retention Time
CRT: Controlled Room Temperature

The stability data indicated that the trilayer tablets remained stable since the impurity profile is within the prescribed limits.

We claim:

1. A layered pharmaceutical composition comprising: a first layer of sustained-release bupropion or its pharmaceutically acceptable salts, a second layer of sustained-release naltrexone or its pharmaceutically acceptable salts, with suitable pharmaceutically acceptable excipients, and an intermediate inert layer that provides a time-controlled disintegration to allow separation of two drug layers and maintain their physical integrity as a single tablet.

2. The layered pharmaceutical composition according to claim 1, wherein the composition comprises first matrix layer of sustained-release bupropion or its pharmaceutically acceptable salts, and a second matrix layer of sustained-release naltrexone or its pharmaceutically acceptable salts, and an intermediate inert layer.

3. The layered pharmaceutical composition according to claim 1, wherein the intermediate inert layer comprises mannitol or glyceryl behenate.

4. The layered pharmaceutical composition according to claim 1, wherein the intermediate inert layer provides the time-controlled disintegration in less than 5 minutes.

5. The layered pharmaceutical composition according to claim 1, wherein the first layer comprises between about 50 mg and about 200 mg of sustained-release bupropion or its pharmaceutically acceptable salts, a second layer comprises between about 2 mg and about 35 mg of sustained-release naltrexone or its pharmaceutically acceptable salts, wherein the rate-controlling agent is contained in the drug layers of the said composition at a concentration of 1-50% W/W.

6. The layered pharmaceutical composition according to claim 1, wherein the first layer comprises between about 50 mg and about 200 mg of sustained-release bupropion or its pharmaceutically acceptable salts, a second layer comprises between about 2 mg and about 35 mg of sustained-release naltrexone or its pharmaceutically acceptable salts, and an intermediate inert layer comprises mannitol or glyceryl behenate, with suitable pharmaceutically acceptable excipients.

7. The layered pharmaceutical composition according to claim 1, wherein the first layer comprises between about 50 mg and about 200 mg of sustained-release bupropion or its pharmaceutically acceptable salts, a second layer comprises between about 2 mg and about 35 mg of sustained-release naltrexone or its pharmaceutically acceptable salts, and an intermediate inert layer, wherein the naltrexone dissolution profile in a dissolution test of USP Apparatus 2 Paddle Method at 50 rpm in a dissolution medium of water is:
a) between 40 to 80% of naltrexone released in one hour;
b) between 60 to 90% of naltrexone released in two hours.
c) at least 99% % of naltrexone released in 8 hours.

8. The layered pharmaceutical composition according to claim 1, wherein the first layer comprises between about 50 mg and about 200 mg of sustained-release bupropion or its pharmaceutically acceptable salts, a second layer comprises between about 2 mg and about 35 mg of sustained-release naltrexone or its pharmaceutically acceptable salts, and an intermediate inert layer, wherein the sustained-release formulation of naltrexone or a pharmaceutically acceptable salt thereof provides an in-vitro release rate of naltrexone in the dissolution test of at least 99% in 8 hours.

9. A method of treating overweight or obesity, comprising a layered pharmaceutical composition, wherein about 180 mg of said sustained-release formulation of bupropion or a pharmaceutically acceptable salt thereof is administered twice daily, and about 16 mg of said sustained-release formulation of naltrexone or a pharmaceutically acceptable salt thereof is administered twice daily.

10. The layered pharmaceutical composition according to claim 1, wherein the pharmaceutical composition is prepared by granulation or pelletization method.
